(19) United States
(12) Patent Application Publication
(10) Pub. No.: US 2018/0250475 A1
(43) Pub. Date: Sep. 6, 2018

(54) PISTON ROD

(71) Applicant: SANOFI, Paris (FR)

(72) Inventors: Anthony Paul MORRIS, Coventry, West Midlands (GB); William MARSH, Buckinghamshire (GB); Matthew Meredith JONES, Warwick, Warwickshire (GB)

(21) Appl. No.: 15/758,453

(22) PCT Filed: Aug. 8, 2016
(86) PCT No.: PCT/EP2016/068857

§ 371 Date: Mar. 8, 2018

(30) Foreign Application Priority Data
Sep. 9, 2015 (EP) 15306372.2

(51) Int. Cl. A61M 5/315

(52) U.S. Cl. CPC A61M 5/31515; A61M 2207/00

(57) ABSTRACT

The disclosure provides a piston rod configured to engage a housing of a drug delivery device, wherein the piston rod comprises an outer thread that corresponds with an inner thread of the housing,
characterized by a support element (that is configured as a supplement thread section of the outer thread,
wherein the support element comprises a lead-in for assembly of the drug delivery device and
wherein the lead-in is distally spaced from another lead-in arranged on the distal outer thread end.

The disclosure further provides a drug delivery device comprising such a piston rod and a method for assembling such a drug delivery device.

### CROSS REFERENCE TO RELATED APPLICATIONS

The present application is the national stage entry of International Patent Application No. PCT/EP2016/068857, filed on Aug. 8, 2016, and claims priority to Application No. EP 15306372.2, filed in on Sep. 9, 2015, the disclosures of which are expressly incorporated herein in entirety by reference thereto.

### TECHNICAL FIELD

The disclosure relates to a piston rod, to a drug delivery device comprising such a piston rod and to a method for assembling such a drug delivery device.

### BACKGROUND DISCLOSURE

In certain types of drug delivery devices, such as pen-type devices, pre-filled cartridges are used. These cartridges are housed in a cartridge holder or housing. To dispense a certain set dose of a medicament contained in such cartridge, the drug delivery device has a dose setting element. During drug delivery, a piston rod coupled to the dose setting element presses against a piston (also commonly referred to as a “bung”, a “stopper”, or a “plunger”) contained within the cartridge in order to dispense the medicament through an attached needle assembly.

The piston rod may be engaged to the housing which accommodates the cartridge. For example, the piston rod is threaded to the housing, wherein the piston rod is rotated with respect to the housing in order to deliver the set dose of medicament. An assembly of a drug delivery device comprising such threaded piston rod may be implemented by an axial insertion of the piston rod into the housing until the corresponding threads abut against each other. After that, the piston rod is initially rotated relative to the housing in order to establish the threaded engagement between the piston rod and the housing.

There remains a need for an improved piston rod that enables an improved thread engagement between the piston rod and the housing with respect to the related art. There remains furthermore a need for a drug delivery device comprising such an improved piston rod and to a method for assembling such a drug delivery device.

### SUMMARY DISCLOSURE

Certain aspects of the present disclosure can be implemented to provide an improved piston rod with a higher efficiency, a drug delivery device comprising such an improved piston rod and to a method for assembling such a drug delivery device.

Certain aspects of the present disclosure can be implemented as a piston rod according to claim 1, by a drug delivery device according to claim 6 and by a method according to claim 10.

Exemplary embodiments of the disclosure are given in the dependent claims.

A piston rod is provided configured to engage a housing of a drug delivery device, wherein the piston rod comprises an outer thread that corresponds with an inner thread of the housing. According to the disclosure, the piston rod comprises a support element that is configured as a supplement thread section of the outer thread, wherein the support element comprises a lead-in for assembly of the drug delivery device and wherein the lead-in is distally spaced from another lead-in arranged on a distal outer thread end.

The support element thus provides an additional contact surface during assembly, thereby increasing the thread engagement between the piston rod and the housing. As a result, an axial length of the drug delivery device may be reduced with respect to the related art due to a possible reduction of windings on a proximal end of the outer thread of the piston rod that engages a distal end of the inner thread of the housing at the end of medicament delivery (last dose of medicament).

The support element may be configured as a projection that extends over an outer piston rod circumference with an angle less than 180 degrees, for example with an angle of 60 degrees.

In an exemplary embodiment, the lead-in is arranged on a proximal end of the support element and the other lead-in is arranged on the distal outer thread end. For example, the lead-ins respectively comprise an edge shape, in particular an edge shape in form of a triangular chamfer. Additionally, there may be also lead-ins on other thread portions of the piston rod and housing that first engage when the piston rod is rotated. Due to the angled contact faces on the threads, when an axial load is applied to the piston rod, a radial load is generated. If there is sufficient engagement in the thread, i. e. something approaching 360°, the radial forces are cancelled out. But if the initial engagement is less than 360° (it is desirable to minimized this to improve the efficiency of the assembly process), the radial load generated on the thread must be balanced by an additional radial contact between the piston rod and the housing, typically between the cylindrical face on the piston rod at the root of the thread and the cylindrical face on the housing at the tip of the thread. This additional radial contact creates friction and reduces the efficiency of the piston rod.

Furthermore, the lead-ins may be angularly offset from one another at an angle of approximately 150 degrees to 180 degrees. Preferably, the angle is about 180 degrees, thus radial forces getting balanced during an initial small rotation of the piston rod with respect to the housing during assembly in order to engage the piston rod and the housing.

In a further exemplary embodiment, a drug delivery device for dispensing a dose of a medicament comprises the provided piston rod and a housing including an inner thread corresponding with the outer thread of the piston rod. Thereby, the inner thread comprises at least one recess whose dimensions at least partially correspond with those of the support element.

The at least one recess enables an easy assembly, wherein the piston rod is moved axially with respect to the housing until the threads abut against each other.

In an exemplary embodiment, the at least one recess engages the support element during assembly. Thus, the support element may pass the recess, thereby allowing the proximal inner thread end to abut the distal outer thread end.

In a further exemplary embodiment, the at least one recess angularly extends with a central angle of approximately 60 degrees. This angle corresponds with those of the support element.

Furthermore, the at least one recess may be arranged within a thread flank of the inner thread part between a proximal inner thread end and a distal inner thread part, wherein the recess is angularly spaced approximately 170 degrees to 200 degrees from the proximal inner thread end. This allows the inner thread to pass the support element during assembly.

According to a further exemplary embodiment, a method for assembly of the drug delivery device comprises the following steps:
inserting the piston rod partially into the housing by axially moving the piston rod in a distal direction with respect to the housing, wherein the lead-in of the support element leads the inner thread between the support element and the outer thread and
rotating the piston rod with respect to the housing after the support element has axially passed the at least one recess.

The assembly of the drug delivery device may be performed as some kind of bayonet mount, wherein the piston rod is initially moved axially with respect to the housing and afterwards rotated with respect to the housing, thereby mechanically engaging the housing. Due to the support element, the piston rod provides an additional contact surface for the inner thread of the housing during assembly. This increases the strength of the thread engagement after the initial rotation of the piston rod and stabilizes the initial rotation.

Further scope of applicability of the present invention will become apparent from the detailed description given hereinafter. However, it should be understood that the detailed description and specific examples, while indicating exemplary embodiments of the invention, are given by way of illustration only, since various changes and modifications within the spirit and scope of the invention will become apparent to those skilled in the art from this detailed description.

### BRIEF DESCRIPTION OF THE FIGURES

The present invention will become more fully understood from the detailed description given herein below and the accompanying drawings which are given by way of illustration only, and thus, are not limitative of the present invention, and wherein:

FIG. 1 is a schematic view of an exemplary embodiment of a drug delivery device comprising a button, a dial grip, a housing and a piston rod,

FIG. 2 is a schematic perspective partly cut-out view of a distal part of an exemplary embodiment of a piston rod,

FIG. 3 is a cross section of a drug delivery device comprising the housing,

FIG. 4 is a schematic perspective view of a proximal thread section and a distal thread section of a thread arranged within the housing,

FIG. 5 is a schematic perspective view of a distal part of an exemplary embodiment of the piston rod and the proximal thread section of the housing during assembly,

FIG. 6 is a schematic perspective view of the distal part of the piston rod and the proximal thread section of the housing during assembly, and

FIG. 7A to 7F are schematic views of a thread engagement between the piston rod and the housing during assembly of the drug delivery device.

Corresponding parts are marked with the same reference symbols in all figures.

### DETAILED DESCRIPTION

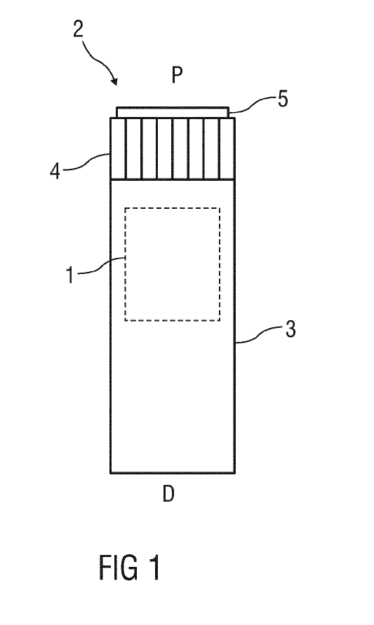

FIG. 1 shows a schematic view of a drug delivery device 2 that may be configured as an injector pen.

The drug delivery device 2 comprises a piston rod 1 inserted into a housing 3, a dial grip 4 and a button 5.

The drug delivery device 2 extends axially between a proximal direction P and a distal direction D. In the present application, the proximal direction P refers to a direction that under use of the drug delivery device 2 is located the furthest away from a drug delivery site of a patient. Correspondingly, the distal direction D refers to a direction that under use of the drug delivery device 2 is located closest to the drug delivery site of the patient.

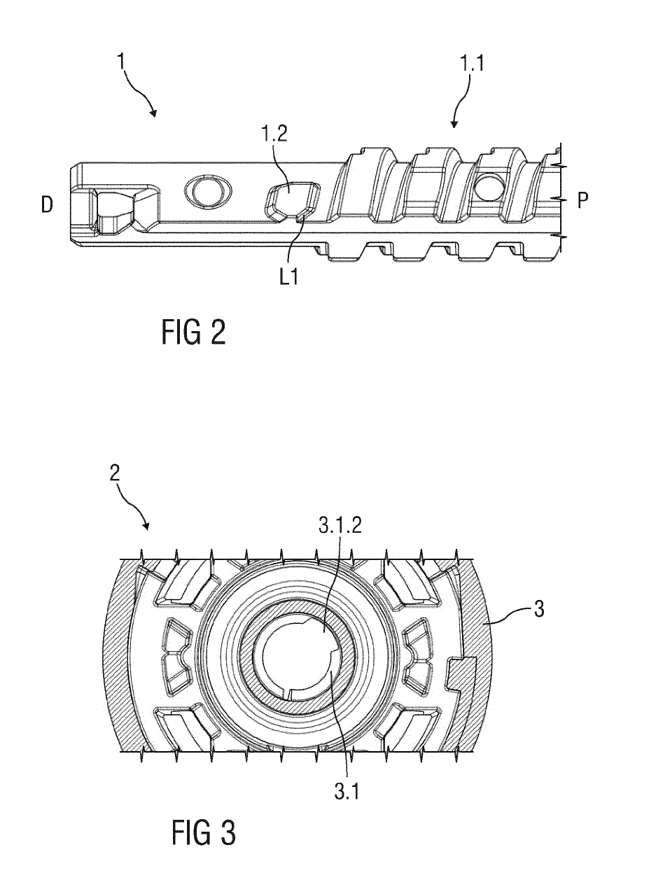

The piston rod 1 and the housing 3 will be described in more detail in the following FIGS. 2 to 7F. The dial grip 4 is axially constrained to the housing 3 and is rotationally constrained to the button 5. The dial grip 4 is a sleeve-like component with a serrated outer skirt. The button 5 forms the proximal end of the drug delivery device 2 and is permanently splined to the dial grip 4.

FIG. 2 schematically shows a distal part of the piston rod 1 in a perspective partly cut-out view.

The piston rod 1 is configured as an elongated member of the drug device 2 which is illustrated in FIGS. 1 and 3. The piston rod 1 may comprise a cylindrical shape and an outer thread 1.1 that may engage a corresponding inner thread 3.1 of the housing 3 (shown in FIG. 3) of the drug delivery device 2.

The piston rod 1 further comprises a support element 1.2 that may be configured as a projection or a thread flank that extends over the outer circumference of the piston rod 1 with a central angle less than 180 degrees, for example with a central angle of 60 degrees. The support element 1.2 is arranged spaced from the outer thread 1.1 in a distal direction D and is adapted to form a blocking member after assembly of the drug delivery device 2 as it is described in more detail in the following FIGS. 3 to 7F.

The outer thread 1.1 may be configured as a well-known external thread with a left-handedness or alternatively with a right-handedness. The outer thread 1.1 comprises a distal outer thread end 1.1.1 and a not shown proximal end, wherein the distal outer thread end 1.1.1 faces the support element 1.2 and the proximal end faces the proximal end of the piston rod 1 in a proximal direction P.

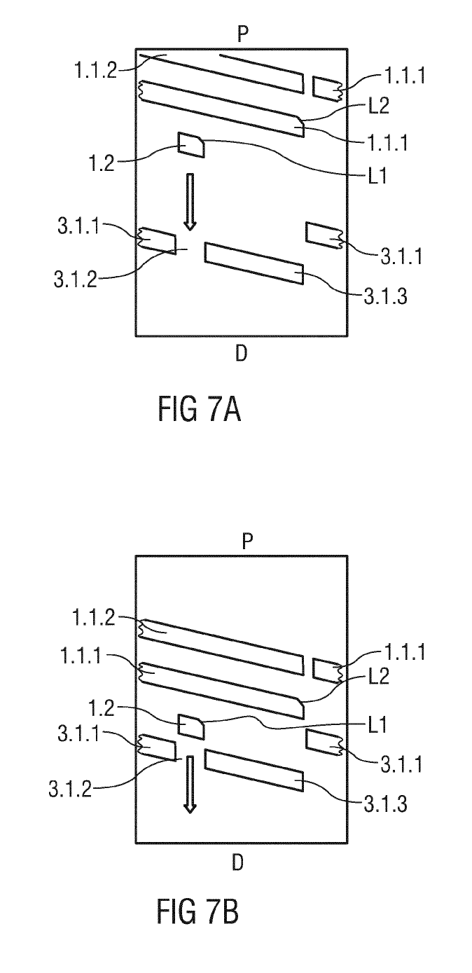

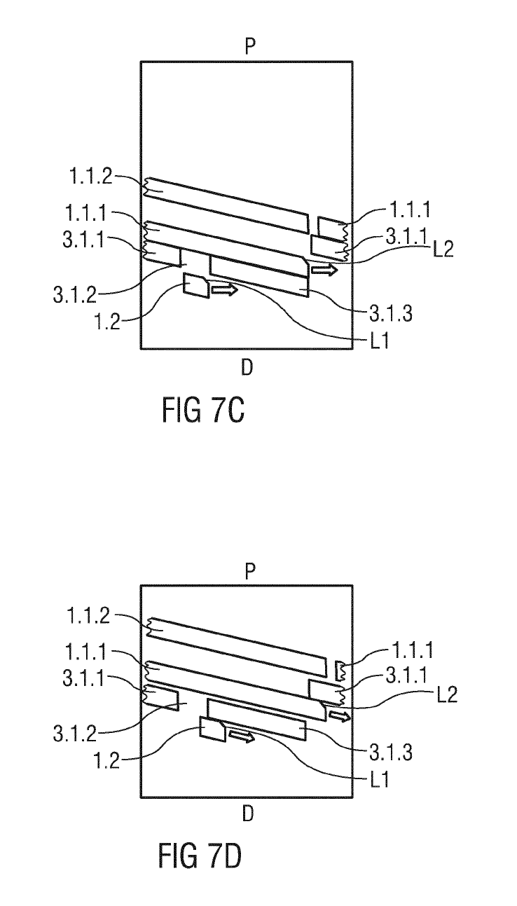

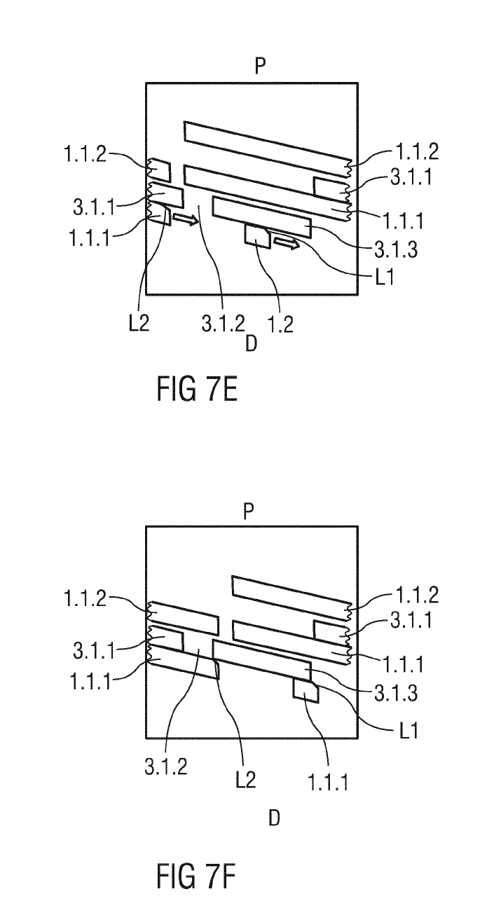

The support element 1.2 and the outer thread 1.1 respectively comprise a lead-in L1, L2 for assembly, for example an edge shape in form of a triangular chamfer, wherein the lead-in L1 of the support element 1.2 is arranged on a proximal end of the support element 1.2 and the second lead-in L2 (illustrated in FIG. 7A) is arranged on the distal outer thread end 1.1.1. The lead-in L1 of the support element 1.2 is adapted to lead a distal inner thread part 3.1.3 of the housing 3 between the support element 1.2 and the outer thread 1.1 during assembly. The lead-in L2 of the outer thread 1.1 is adapted to engage the corresponding inner thread 3.1 on the first or initial rotation of the piston rod 1 relative to the housing 3. Hence, the support element 1.2 provides a second contact surface for the inner thread 3.1 during the initial rotation, thereby improving the thread engagement between the piston rod 1 and the housing 3 with respect to the related art.

FIG. 3 shows a cross section of the housing 3.

The housing 3 comprises the inner thread 3.1 as described already above. As it is illustrated by the cross section, the inner thread 3.1 comprises a recess 3.1.2 that extends angularly with a central angle of approximately 60 degrees over an inner circumference of the housing 3, thereby corresponding with the central angle of the support element 1.2 at which it extends over the outer circumference of the piston rod 1. The recess 3.1.2 is arranged within a thread flank of inner thread part 3.1 and that is angularly spaced approximately 170 degrees to 200 degrees from the proximal inner thread end 3.1.1. The recess 3.1.2 allows the support element 1.2 to pass through during assembly, thereby enabling an axial movement of the piston rod 1 with respect to the housing 3 in order to engage the outer thread 1.1 and the inner thread 3.1.

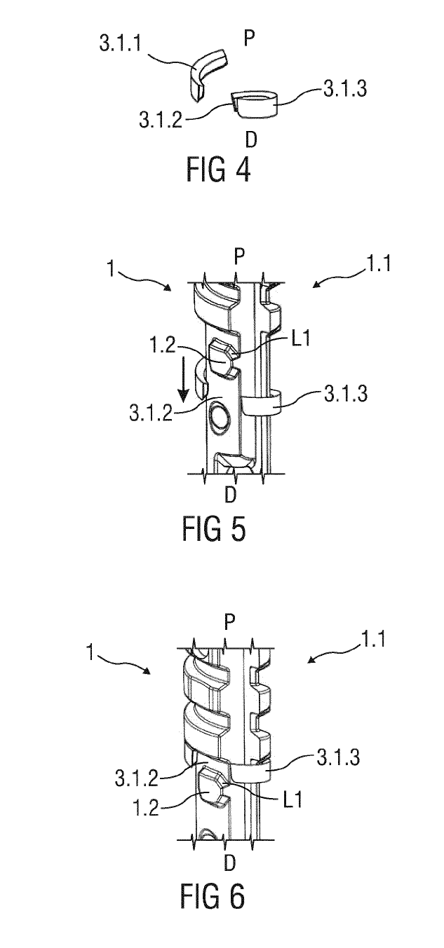

FIG. 4 shows a perspective view of the proximal inner thread end 3.1.1, the recess 3.1.2 and a distal inner thread part 3.1.3 that is arranged immediately distally behind the recess 3.1.2.

FIG. 5 shows the distal part of the piston rod 1 in a perspective view and the distal inner thread part 3.1.3 during assembly. Here, the piston rod 1 is inserted into the housing 3 by moving the piston rod 1 in the distal direction D with respect to the housing 3 (the assembly direction is marked with an arrow). Referring to the assembly step illustrated in the present figure, the distal inner thread part 3.1.3 is located distally behind the support element 1.2.

FIG. 6 shows the distal part of the piston rod 1 in a perspective view and the distal inner thread part 3.1.3 during assembly immediately before the initial rotation of the piston rod 1 relative to the housing 3. The distal inner thread part 3.1.3 has axially passed the support element 1.2 due to the recess 3.1.2 and distally abuts the distal outer thread end 1.1.1. The assembly of the drug delivery device 2 regarding the piston rod 1 and the housing 3 is further described in the following FIGS. 7A to 7F.

FIGS. 7A to 7F show simplified schematic views of the outer thread 1.1 and the inner thread 3.1 during assembly of the drug delivery device 2. In particular shown are: the support element 1.2 and the distal outer thread end 1.1.1 followed by a proximal outer thread part 1.1.2 which represents a second winding of the outer thread 1.1 immediately following the distal outer thread end 1.1.1 in the proximal direction P; and a part of the inner thread 3.1 comprising the proximal inner thread end 3.1.1 and the distal inner thread part 3.1.3 which represents a second winding of the inner thread 3.1 immediately following the proximal inner thread end 3.1.1 in the distal direction D. Further outer thread parts may be added to the outer thread 1.1 in the proximal direction P, and each outer thread part may be divided into further parts, for example to aid manufacturing.

FIG. 7A shows a first assembly step, wherein the outer thread 1.1 and the inner thread 3.1 are not engaged and the distal inner thread part 3.1.3 as well as the proximal inner thread end 3.1.1 is located distally behind the support element 1.2.

FIG. 7B shows a second assembly step, wherein the piston rod 1 has moved distally with respect to the housing 3 (indicated by an arrow illustrated in FIG. 7A). The distal inner thread part 3.1.3 is still located distally behind the support element 1.2 with an axial distance less than the distance illustrated in FIG. 7A. The proximal inner thread end 3.1.1 is at least partially located between the support element 1.2 and the outer thread 1.1. Furthermore, the proximal inner thread end 3.1.1 is angularly spaced from support element 1.2. Thus, the distal inner thread part 3.1.3 has not yet contacted the lead-in L1 of the support element 1.2.

FIG. 7C shows a third assembly step, wherein the piston rod 1 has moved further distally with respect to the housing 3 (indicated by an arrow illustrated in FIG. 7B). The support element 1.2 has passed the distal inner thread part 3.1.3 and thus the distal inner thread part 3.1.3 distally abuts the distal outer thread end 1.1.1, wherein the recess 3.1.2 and the support element 1.2 are angularly aligned to each other with respect to an axis parallel to the longitudinal extension of the piston rod 1. As it can be seen in the present illustration, the lead-ins L1, L2 are angularly spaced from each other at an angle of approximately 180 degrees. Thus, radial forces during further assembly steps are balanced and an additional radial contact between the piston rod 1 and the housing 3 is reduced.

FIG. 7D shows a fourth assembly step, wherein the piston rod 1 has rotated counter clockwise with respect to the housing 3 (indicated by an arrow illustrated in FIG. 7C). Thus, the recess 3.1.2 and the support element 1.2 get angularly spaced from each other and the proximal inner thread end 3.1.1 moves between the distal outer thread end 1.1.1 and the proximal outer thread part 1.1.2. The piston rod 1 and the housing 3 are now engaged to each other.

FIG. 7E shows a fifth assembly step, wherein the piston rod 1 has been further rotated counter clockwise with respect to the housing 3 (indicated by an arrow illustrated in FIG. 7D). The thread engagement between the piston rod 1 and the housing 3 is increased, wherein the proximal inner thread end 3.1.1 engages the distal outer thread end 1.1.1 and the proximal outer thread part 1.1.2.

FIG. 7F shows a sixth assembly step, wherein the piston rod 1 has been further rotated counter clockwise with respect to the housing 3 (indicated by an arrow illustrated in FIG. 7E). The thread engagement between the piston rod 1 and the housing 3 is further increased and the recess 3.1.2 is bridged by the distal outer thread end 1.1.1.

As it can be seen by the illustrated sequence from FIG. 7A to 7F, a thread engagement with an acceptable efficiency may be achieved by an initial rotation of less than 180 degrees and preferably less than 60 degrees. Contrary to this, a piston rod 1 with a single outer thread 1.1 without the support element 1.2 would require a rotation of approximately 360 degrees for a thread engagement with an acceptable efficiency. As a result, a length of the drug delivery device 2 may be reduced with respect to the related art, thereby reducing the number of windings at a proximal end of the piston rod 1 and thus, reducing an initial gap between the piston rod 1 and a bearing (not shown). The bearing is a component of the drug delivery device 2 that is axially constrained to the piston rod 1 and that acts on the piston within the medicament cartridge. It is axially clipped to the piston rod 1, but free to rotate.

Furthermore, the drug delivery device 2 described before enables an improved strength of the thread engagement between the piston rod 1 and the housing 3 after the initial rotation of less than approximately 60 degrees. Moreover, a time for assembly will be reduced with respect to the related art.

The terms “drug” or “medicament” are used herein to describe one or more pharmaceutically active compounds. As described below, a drug or medicament can include at least one small or large molecule, or combinations thereof, in various types of formulations, for the treatment of one or more diseases. Exemplary pharmaceutically active compounds may include small molecules; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more of these drugs are also contemplated.

The term “drug delivery device” shall encompass any type of device or system configured to dispense a drug into a human or animal body. Without limitation, a drug delivery device may be an injection device (e.g., syringe, pen injector, auto injector, large-volume device, pump, perfusion system, or other device configured for intraocular, subcutaneous, intramuscular, or intravascular delivery), skin patch (e.g., osmotic, chemical, micro-needle), inhaler (e.g., nasal or pulmonary), implantable (e.g., coated stent, capsule), or feeding systems for the gastro-intestinal tract. The presently described drugs may be particularly useful with injection devices that include a needle, e.g., a small gauge needle.

The drug or medicament may be contained in a primary package or “drug container” adapted for use with a drug delivery device. The drug container may be, e.g., a cartridge, syringe, reservoir, or other vessel configured to provide a suitable chamber for storage (e.g., short- or long-term storage) of one or more pharmaceutically active compounds. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of a drug formulation (e.g., a drug and a diluent, or two different types of drugs) separately, one in each chamber. In such instances, the two chambers of the dual-chamber cartridge may be configured to allow mixing between the two or more components of the drug or medicament prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drug delivery devices and drugs described herein can be used for the treatment and/or prophylaxis of many different types of disorders. Exemplary disorders include, e.g., diabetes mellitus or complications associated with diabetes mellitus such as diabetic retinopathy, thromboembolism disorders such as deep vein or pulmonary thromboembolism. Further exemplary disorders are acute coronary syndrome (ACS), angina, myocardial infarction, cancer, macular degeneration, inflammation, hay fever, atherosclerosis and/or rheumatoid arthritis.

Exemplary drugs for the treatment and/or prophylaxis of diabetes mellitus or complications associated with diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the term “derivative” refers to any substance which is sufficiently structurally similar to the original substance so as to have substantially similar functionality or activity (e.g., therapeutic effectiveness).

Exemplary insulin analogues are Gly(A21), Arg(B31), Arg(B32) human insulin (insulin glargine); Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Exemplary insulin derivatives are, for example, B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin; B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin. Exemplary GLP-1, GLP-1 analogues and GLP-1 receptor agonists are, for example: Lixisenatide/AVE0010/ZP10/Lyxumia, Exenatide/Exendin-4/Byetta/Bydureon/ITCA 650/AC-2993(a 39 amino acid peptide which is produced by the salivary glands of the Gila monster), Liraglutide/Victoza, Semaglutide, Taspoglutide, Syncria/Albiglutide, Dulaglutide, rExendin-4, CJC-1134-PC, PB-1023, TTP-054, Langlenatide/HM-11260C, CM-3, GLP-1 Eligen, ORMD-0901, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, TT-401, BHM-034. MOD-6030, CAM-2036, DA-15864, ARI-2651, ARI-2255, Exenatide-XTEN and Glucagon-Xten.

An exemplary oligonucleotide is, for example: mipomersen/Kynamro, a cholesterol-reducing antisense therapeutic for the treatment of familial hypercholesterolemia.

Exemplary DPP4 inhibitors are Vildagliptin, Sitagliptin, Denagliptin, Saxagliptin, Berberine.

Exemplary hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Exemplary polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20/Synvisc, a sodium hyaluronate.

The term “antibody”, as used herein, refers to an immunoglobulin molecule or an antigen-binding portion thereof. Examples of antigen-binding portions of immunoglobulin molecules include F(ab) and F(ab′)₂ fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region.

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present disclosure include, for example, Fab fragments, F(ab′)2 fragments, scFv (single-chain Fv) fragments, linear antibodies, monospecific or multispecific antibody fragments such as bispecific, trispecific, and multispecific antibodies (e.g., diabodies, triabodies, tetrabodies), minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Exemplary antibodies are anti PCSK-9 mAb (e.g., Alirocumab), anti IL-6 mAb (e.g., Sarilumab), and anti IL-4 mAb (e.g., Dupilumab).

The compounds described herein may be used in pharmaceutical formulations comprising (a) the compound(s) or pharmaceutically acceptable salts thereof, and (b) a pharmaceutically acceptable carrier. The compounds may also be used in pharmaceutical formulations that include one or more other active pharmaceutical ingredients or in pharmaceutical formulations in which the present compound or a pharmaceutically acceptable salt thereof is the only active ingredient. Accordingly, the pharmaceutical formulations of the present disclosure encompass any formulation made by admixing a compound described herein and a pharmaceutically acceptable carrier.

Pharmaceutically acceptable salts of any drug described herein are also contemplated for use in drug delivery devices. Pharmaceutically acceptable salts are for example acid addition salts and basic salts. Acid addition salts are e.g. HCl or HBr salts. Basic salts are e.g. salts having a cation selected from an alkali or alkaline earth metal, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are known to those of skill in the arts.

Pharmaceutically acceptable solvates are for example hydrates or alkanolates such as methanolates or ethanolates.

Those of skill in the art will understand that modifications (additions and/or removals) of various components of the substances, formulations, apparatuses, methods, systems and embodiments described herein may be made without departing from the full scope and spirit of the present invention, which encompass such modifications and any and all equivalents thereof.

### LIST OF REFERENCES

1 piston rod
1.1 outer thread
1.1.1 distal outer thread end
1.1.2 proximal outer thread part
1.2 support element
2 drug delivery device
3 housing
3.1 inner thread
3.1.1 proximal inner thread end
3.1.2 recess
3.1.3 distal inner thread part
4 dial grip
5 button
D distal direction
L1, L2 lead-in
P proximal direction

1-10. (canceled)

11. A piston rod configured to engage a housing of a drug delivery device, the piston rod comprising:
an outer thread that corresponds with an inner thread of the housing; and
a support element that is configured as a supplement thread section of the outer thread, wherein the support element comprises a lead-in for assembly of the drug delivery device, and wherein the lead-in is distally spaced from another lead-in arranged on the distal outer thread end.

12. The piston rod according to claim 11,
wherein the support element is configured as a projection that extends over an outer piston rod circumference with an angle less than 180 degrees.

13. The piston rod according to claim 11,
wherein the lead-in (L1) is arranged on a proximal end of the support element.

14. The piston rod according to claim 13,
wherein the lead-in and the other lead-in respectively comprise an edge shape.

15. The piston rod according to claim 13,
wherein the lead-in and the other lead-in are angularly offset from one another at an angle of approximately 180 degrees.

16. A drug delivery device for dispensing a dose of a medicament, the drug delivery device comprising:
a piston rod configured to engage a housing of a drug delivery device, the piston rod comprising:
an outer thread that corresponds with an inner thread of the housing; and
a support element that is configured as a supplement thread section of the outer thread, wherein the support element comprises a lead-in for assembly of the drug delivery device, and wherein the lead-in is distally spaced from another lead-in arranged on the distal outer thread end; and
the housing including the inner thread corresponding with the outer thread of the piston rod, wherein the inner thread comprises at least one recess whose dimensions at least partially correspond with those of the support element.

17. The drug delivery device according to claim 16,
wherein the at least one recess engages the support element during assembly.

18. The drug delivery device according to claim 16,
wherein the at least one recess angularly extends with a central angle of approximately 60 degrees.

19. The drug delivery device according to claim 16,
wherein the at least one recess is arranged within a thread flank of the inner thread and is angularly spaced approximately 170 degrees to 200 degrees from a proximal inner thread end.

20. The drug delivery device according to claim 16,
wherein the support element is configured as a projection that extends over an outer piston rod circumference with an angle less than 180 degrees.

21. The drug delivery device according to claim 16,
wherein the lead-in (L1) is arranged on a proximal end of the support element.

22. The drug delivery device according to claim 21,
wherein the lead-in and the other lead-in respectively comprise an edge shape.

23. The drug delivery device according to claim 21,
wherein the lead-in and the other lead-in are angularly offset from one another at an angle of approximately 180 degrees.

24. A method for assembling a drug delivery device comprising:
inserting a piston rod partially into a housing by axially moving the piston rod in a distal direction with respect to the housing, the piston rod comprising:
an outer thread that corresponds with an inner thread of the housing; and
a support element that is configured as a supplement thread section of the outer thread, wherein the support element comprises a lead-in for assembly of the drug delivery device, and wherein the lead-in is distally spaced from another lead-in arranged on the distal outer thread end,
wherein the lead-in of the support element leads the inner thread between the support element and the outer thread, and
rotating the piston rod with respect to the housing after the support element has axially passed the at least one recess.
